(19) United States
(12) Patent Application Publication
(10) Pub. No.: US 2016/0346324 A1
(43) Pub. Date: Dec. 1, 2016

(54) Method of Inactivating Bacterial Lipases Using Oxidative Chlorine Species

(71) Applicant: NovaBay Pharmaceuticals, Inc., Emeryville, CA (US)

(72) Inventors: Arthur B. Epstein, Phoenix, AZ (US); Lu Wang, Moraga, CA (US); Dmitri Debabov, Emeryville, CA (US); Kathryn Najafi, Emeryville, CA (US); Ramin Najafi, Emeryville, CA (US)

(73) Assignee: NovaBay Pharmaceuticals, Inc., Emeryville, CA (US)

(21) Appl. No.: 15/167,435

(22) Filed: May 27, 2016

Related U.S. Application Data
(60) Provisional application No. 62/168,666, filed on May 29, 2015.

(51) Int. Cl. A61K 33/20; A61K 47/02; A61K 45/06

(52) U.S. Cl. CPC A61K 33/20; A61K 45/06; A61K 47/02

(57) ABSTRACT

Lipases produced by bacteria break down tear lipids and cause or exacerbate discomfort due to meibomian gland dysfunction, blepharitis or dry eye. In one embodiment, the present invention provides methods of treating and prevention further discomfort by inactivating lipases through use chlorinated solution and or their derivatives.

### RELATED APPLICATION

This application claims the benefit of U.S. Provisional Application No. 62/168,666, filed May 29, 2015.

### BACKGROUND OF THE INVENTION

Meibomian gland dysfunction (MGD), which is sometimes called posterior blepharitis, is a frequent cause of inflammation of the eyelids and ocular surface. MGD is caused by obstruction or abnormal secretion of meibomian glands that run radially within both upper and lower eyelids. Meibomian glands normally secrete meibum, which forms the complex lipid-rich layer of the tear film. Meibum stabilizes the tear structure, reduces evaporation, and can serve as a carbon source for bacteria colonizing lid surfaces.

Meibomian gland secretion in normal people mainly consists of neutral sterols and wax esters (which are non-polar lipids), with lesser amounts of polar lipids (free fatty acids), diesters, triesters, triglycerides and free sterols. Many changes in meibomian lipid composition, such as increased mono-unsaturated fatty acids and different fatty acid compositions have been documented to contribute to abnormal lipid behaviors and clinical symptoms.

Abnormal meibum has a higher melting temperature, which results in thicker meibum, ductal plugging, stagnation and pouting of the meibomian gland orifices. Excessive amounts of bacteria on the lid surfaces can produce sufficient lipases, fat and oil-reducing enzymes which are thought to degrade the lipid. Inactivating lipases successfully brings about improvement in lipid ordering, contributing to differences in the phase transition temperature of meibum. Due to this change, relief in meibomian gland orifice plugging and improvement in the lipid properties of the meibum gland secretion can be demonstrated (Qiao 2013). MGD may alter the corneal reflectivity, impairing vision.

Lipases (EC 3.1.1.3 triacylglycerol acylhydrolase) are a group of water soluble enzymes, which exhibit the ability of acting at the interface between aqueous and organic phases. They primarily catalyze the hydrolysis of ester bonds in water insoluble lipid substrates. However, some lipases are also able to catalyze the processes of esterification, interesterification, transesterification, acidolysis, aminolysis and may show enantioselective properties.

Lipases are of plant, animal, and microbial origin, but only bacterial and fungal lipases such as: Candida Antarctica (Novozym 435), Candida Rugosa (Lipase AY), Pseudomonascepacia (Lipase PS), Pseudomonas fluorescens (Lipase AK), Pseudomonas aeruginosa, and Thermomyces lanuginose (Lipozime TL), among others are produced at industrial scale (Stoytcheva et al., 2012).

Dougherty McCulley (1986) cultured eyelids and conjunctivae of 36 normal individuals and 60 patients from six clinical groups of chronic blepharitis for aerobic and anaerobic bacteria. The most common species isolated were coagulase-negative staphylococci (C-NS) and Propionibacterium acnes. All strains of these species, and all Staphylococcus aureus strains isolated were tested for the ability to break down triglycerides, cholesterol esters and fatty waxes. Each strain was incubated independently with appropriate substratesin nutrient media. Each medium was then extracted and assayed for the presence of substrate hydrolysis products by thin-layer chromatography. The percentage of strains capable of hydrolyzing a particular substrate was determined for each individual. S. aureus was a consistent and strong lipase producer, able to hydrolyze all three substrates. P. acnes was able to hydrolyze triolein and behenyl oleate but not cholesteryl oleate.

No differences were observed among groups for P. acnes or S. aureus. C-NS showed a high degree of strain variability. Eighty-three percent of C-NS strains could hydrolyze triolein, 82% behenyl oleate and 40% cholesteryl oleate. Significant group differences were seen in the percentage of lipase positive C-NS strains isolated per individual. Patients in the mixed staphylococcal/seborrheic, meibomian seborrheic, secondary meibomitis, and the meibomian kerato conjunctivitis (MKC) groups harbored significantly more C-NS strains capable of hydrolyzing cholesteryl oleate than did normal individuals. Patients in the meibomian seborrheic, secondary meibomitis, and MKC groups harbored significantly more C-NS strains capable of hydrolyzing behenyl oleate than did normal people. No group differences were seen among groups with triolein hydrolyzing C-NS strains.

### DESCRIPTION OF THE BACKGROUND ART

U.S. Pat. No. 8,022,027 relates to a composition comprising: (i) a lipase; and (ii) a bleach catalyst that is capable of accepting an oxygen atom from a peroxy acid and transferring the oxygen atom to an oxidizeable substrate.

U.S. Pat. No. 6,133,220 discloses detergent compositions comprising lipase variant D96L of the native lipase derived from Humicola lanuginosa present at a level of from 50 LU to 8500 LU per liter wash solution. Additional optional detergent ingredients that can be included in the detergent compositions of the present invention include bleaching agents such as PB1, PB4 and percarbonate with a particle size of 400-800 microns. These bleaching agent components can include one or more oxygen bleaching agents and, depending upon the bleaching agent chosen, one or more bleach activators. A category of bleaching agents that can be used encompasses the halogen bleaching agents. Examples of hypohalite bleaching agents, for example, include trichloro isocyanuric acid and the sodium and potassium dichloro isocyanurates and N-chloro and N-bromo alkane sulphonamides. Such materials are normally added at 0.5-10% by weight of the finished product, preferably 1-5% by weight. Using these levels of lipase delivers an improved whiteness maintenance on fabrics.

United States Patent Publication No. 20110280854 discloses compositions and methods for treating or preventing E. coli infections. The compositions can be formulated as pharmaceutical compositions or as disinfectants, sanitizers, detergents or antiseptics, and can be used to eradicate or reduce E. coli populations and thereby treat or prevent infection by E. coli. The compositions include one or more digestive enzymes, e.g., one or more protease, lipases, and amylases. As reported in the publication, a disinfectant or sanitizer as described therein can include one or more digestive enzymes, and can optionally include other active and inactive ingredients, including stabilizers (e.g., enzyme stabilizers), other disinfectants known to those having ordinary skill in the art, formulation excipients, colorants, perfumes, etc. Additional active or inactive ingredients may be selected to include in a disinfectant. Examples of additional disinfectants include: sources of active chlorine (i.e., hypochlorites, chloramines, dichloroisocyanurate and trichloroisocyanurate, wet chlorine, chlorine dioxide etc.). Methods of use of the compositions are also provided.

United States Patent Publication No. 20110280853 discloses compositions and methods for treating or preventing S. aureus infections. As reported in the publication, the compositions can be formulated as pharmaceutical compositions or as disinfectants, sanitizers, detergents or antiseptics, and can be used to eradicate or reduce S. aureus populations and thereby treat or prevent infection by S. aureus. The compositions include one or more digestive enzymes, e.g., one or more protease, lipases and amylases. A disinfectant or sanitizer as described can include one or more digestive enzymes, and can optionally include other active and inactive ingredients, including stabilizers (e.g., enzyme stabilizers), other disinfectants known to those having ordinary skill in the art, formulation excipients, colorants, perfumes, etc. One having ordinary skill in the art can select the additional active or inactive ingredients to include in a disinfectant. Examples of additional disinfectants include: sources of active chlorine (i.e., hypochlorites, chloramines, dichloroisocyanurate and trichloroisocyanurate, wet chlorine, chlorine dioxide etc.). Methods of use of the compositions are also provided.

U.S. Pat. No. 5,856,167 discloses a protease obtained from Bacillus sp., DSM 8473, which has improved hypochlorite stability as compared to other known proteases. The protease is suitable as a detergent additive and may be used singly or combined with other know enzymes in detergent compositions. A process for washing soiled fabric with detergent compositions containing the hypochlorite stable protease is also disclosed.

### SUMMARY OF THE INVENTION

In one embodiment, the present application discloses a method for the treatment or the prevention of blepharitis, meibomian gland dysfunction, or dry eye associated with lipases in a patient in need thereof, comprising an administration of a therapeutically effective amount of a pharmaceutical composition comprising hypochlorous acid, a hypochlorite salt or a mixtures thereof, to inactivate lipases.

In one aspect of the above method, the hypochlorous acid, hypochlorite salt or a mixtures thereof is at a concentration of 0.005% to 0.05% in an aqueous saline solution. In another aspect of the method, the concentration of the hypochlorous acid, hypochlorite salt or a mixtures thereof is 0.005% to 0.05% in a saline solution at a pH range of 3 to 9. In another aspect, the pH range is 3.5 to 4.5.

In another aspect of the above method, the concentration of the hypochlorous acid, hypochlorite salt or a mixtures thereof is 0.01% in a saline solution at a pH of 4. In another aspect, the hypochlorite salt is NaOCl. In another aspect of the method, the composition comprises hypochlorous acid at a concentration of 0.01% in a saline solution at pH 4. In yet another aspect of the above methods, the pharmaceutical composition further comprises a disinfectant selected from the group consisting of chloramines, dichloroisocyanurate, trichloroisocyanurate, wet chlorine, chlorine dioxide and mixtures thereof.

In another aspect of the above methods, the lipases are from Burkholderia cepacia, Pseudomonas fluorescens, Thermus thermophilus, Talaromyces flavus and Burkholderia species. In another aspect, the lipases are inactivated in less than 10 minutes, less than 5 minutes, less than 2 minutes or less than 1 minute. In another aspect, the method reduces the activity of the lipases in the patient. In another aspect, the method reduces the activity of the lipases on human cells or tissues.

In another embodiment, the application discloses a method for reducing or eliminating the activity of lipases associated with blepharitis, meibomian gland dysfunction, or dry eye in a patient, the method comprising the administration of a therapeutically effective amount of a pharmaceutical composition comprising hypochlorous acid, a hypochlorite salt or mixtures thereof, to inactivate lipases. In one aspect of the method, the inactivation of lipases is from bacterial species that are commensal (normal) skin flora or bacteria recovered from blepharitis, meibomian gland dysfunction or dry eye, or a combination thereof.

In one aspect of the above method, the hypochlorous acid, hypochlorite salt or a mixtures thereof is at a concentration of 0.005% to 0.05% in an aqueous saline solution. In another aspect, the concentration of the hypochlorous acid, hypochlorite salt or a mixtures thereof is 0.005% to 0.05% in a saline solution at a pH range of 3 to 9. In another aspect, the concentration of the hypochlorous acid, hypochlorite salt or a mixtures thereof is 0.01% in a saline solution at a pH of 4. In another aspect of the above method, the hypochlorite salt is NaOCl.

In one variation of the above method, the composition comprises hypochlorous acid at a concentration of 0.01% in a saline solution at pH 4. In another variation of the method, the pharmaceutical composition further comprises a disinfectant selected from the group consisting of chloramines, dichloroisocyanurate, trichloroisocyanurate, wet chlorine, chlorine dioxide and mixtures thereof.

In another aspect of the above method, the lipases are from Burkholderia cepacia, Pseudomonas fluorescence, Thermus thermophilus, Talaromyces flavus and Burkholderia species. In another aspect, the method reduces the activity of the lipases on human cells or tissues.

In one variation of the above method, the lipases are inactivated in less than 10 minutes, less than 5 minutes, less than 2 minutes or less than 1 minute. In another variation, the method reduces the activity of the lipases in the patient.

### BRIEF DESCRIPTION OF THE FIGURES

FIG. 1 is a representative graph depicting inactivation of Talaromyces flavus lipase by 0.001% HOCl.

FIG. 2 is a representative graph depicting inactivation of Pseudomonas fluorescens lipase by 0.009% HOCl.

FIG. 3 is a representative graph depicting inactivation of Burkholderia cepacia lipase by 0.009% HOCl.

FIG. 4 is a representative graph depicting a 0.01% HOCl solution that inactivates lipase, while sterile water does not.

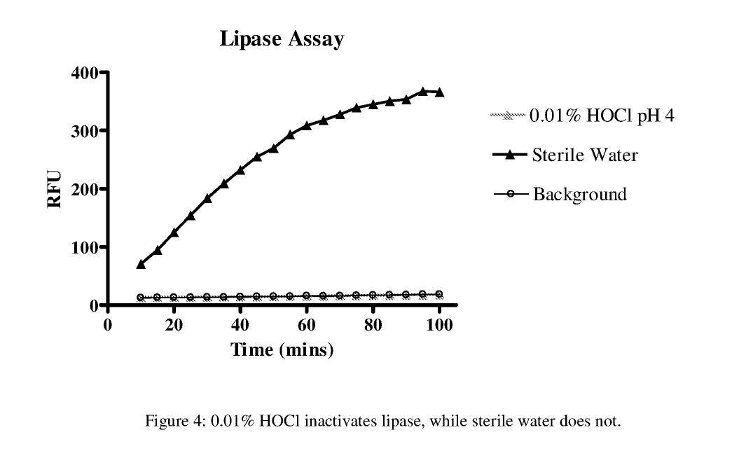

### EXPERIMENTS

Lipases from Burkholderia cepacia, Pseudomonas fluorescens, Thermus thermophilus, Talaromyces flavus and Burkholderia species were obtained from Sigma (Catalog Numbers 62309, 28602, L3419, L3294 and 75577, respectively) and were diluted into sterile water for testing.

Activity of 0.01% HOCl in saline pH 4 against Burkholderia cepacia lipase was determined as follows. 0.01% HOCl was added to a solution of 2 mg/mL of Pseudomonas cepacia lipase (Sigma Aldrich®, St. Louis, Mo., USA). After an hour of incubation at 37° C., the lipase-HOCl solutions were diluted 500-fold into lipase buffer. The lipase activity was determined using the Lipase Activity Assay Kit III (Sigma Aldrich®, St. Louis, Mo., USA).

Alternative procedure: Activity of 0.01% HOCl in saline pH 4 against each lipase was determined as follows. 10 μL of 0.01% HOCl was added to 90 μL of 2 mg/mL lipase (final concentration of HOCl 0.001%). After one hour of incubation at 37° C., the lipase-HOCl solutions were diluted 500-fold into lipase buffer. Alternatively, 90 μL of 0.01% HOCl was added to 10 μL solution of 2 mg/mL lipase (final concentration of HOCl 0.009%) and after an hour of incubation at 37° C., the lipase-HOCl solutions were diluted 50-fold into lipase buffer. The lipase activity was determined using the Lipase Activity Assay Kit III (Sigma Aldrich®, St. Louis, Mo., USA).

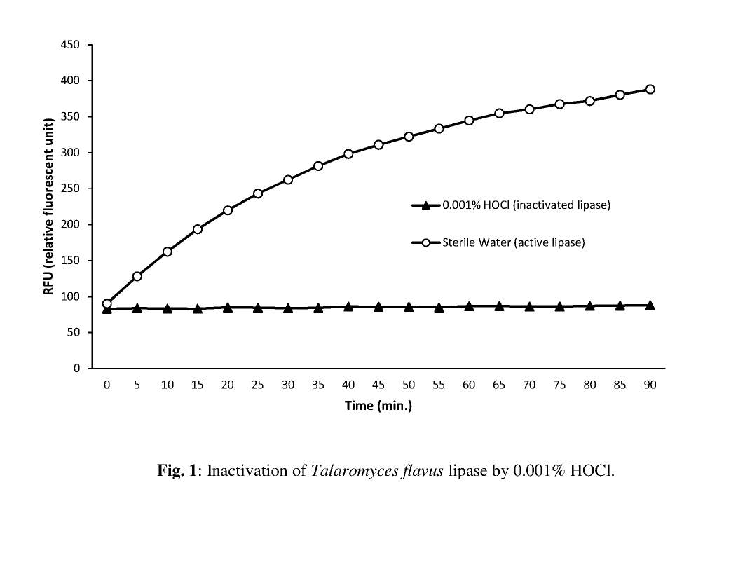

A SpectraMax® M5 Microplate Reader was used to continuously incubate the microtiter plate at 37° C. and measure the fluorescence (λex/λex=529/600 nm) every 5 minutes for 1.5 hours after an initial 10-minute incubation period. The results are shown on FIG. 1. In the presence of 0.01% HOCl, the bacterial lipase was completely inactivated (i.e., not distinguishable from no added lipase).

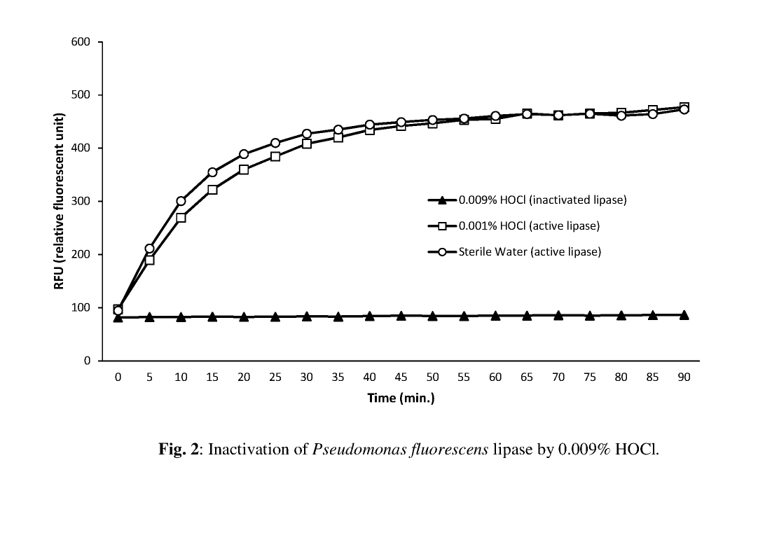

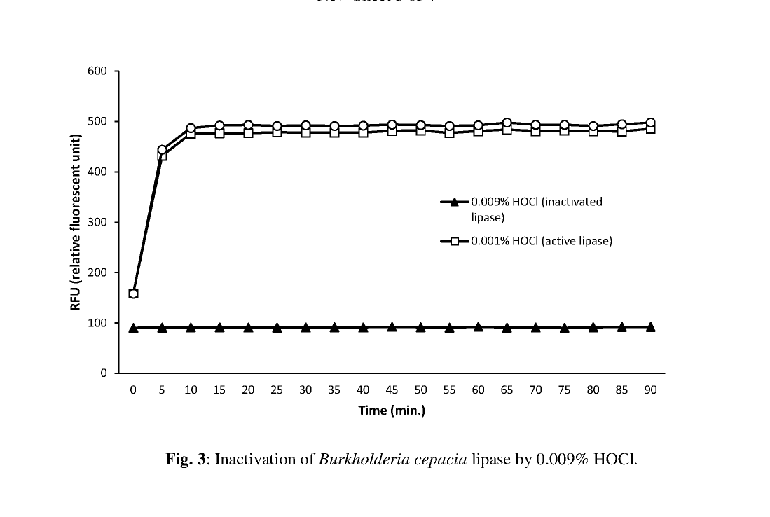

Alternative procedure without a 10 minutes incubation: A SpectraMax® M5 Microplate Reader was used to continuously incubate the microtiter plate at 37° C. and measure the fluorescence (λex/λex=529/600 nm) every 5 minutes for 1.5 hours with no incubation period. The results are shown on FIG. 1. In the presence of 0.01% HOCl, the bacterial lipase was completely inactivated (i.e., not distinguishable from no added lipase). The results are shown on FIGS. 1, 2, and 3. In the presence of 0.001% HOCl, the Talaromyces flavus lipase was completely inactivated (i.e., not distinguishable from no added lipase, see FIG. 1) while the other lipases were still active. In the presence of 0.009% HOCl, all lipases were inactivated (FIGS. 2, 3).

Activity of lipases from Pseudomonas fluorescens, Thermusthermophilus, flavus and Burkholderia are determined using methods similar to the one described above for Burkholderia cepacia lipase. The results of these experiments demonstrate the lack of consistency as it pertains to various concentrations of HOCl and its ability to inactive lipases. Out of 3 lipases, one lipase was inactivated at 0.001% HOCl while the others were not. All lipases were then inactivated at 0.009% HOCl. This was an unexpected result.

Lipase activity from P. aeruginosa, S.aureus and Staphylococcus epidermidis are tested using assays described below.

Methods of lipase activity quantification involve volumetry, spectrometry, radioactive assays, immunoassays, conductimetry, chromatography and biosensors (Stoytcheva et al., 2012).

Merck describes a fluorimetric method where lipase activity is determined using a coupled enzyme reaction, which results in the generation of methylresorufin (lex=529/lem=600 nm) proportional to the enzymatic activity present. One unit of lipase is the amount of enzyme that will generate 1.0 mml of methylresorufin from the substrate per minute at 37° C. (Lipase Activity Assay Kit III Catalog Number MAK048). The lipase stock was treated with equal part of the test product. The control consisted of equal parts of the lipase stock and sterile water. Both were incubated at 37° C. for 1 hour.

The Lipase Activity Assay Kit III (Sigma MAK048) consisted of materials for a standard, a positive control and background control. Once all the controls and the standard were set-up into a 96-well microtiter plate according to the provided instructions, 2 μL of the lipase treatments were diluted into 998 μL of the provided Lipase Assay Buffer. In the microtiter plate, 2 μL of this dilution was added to 48 μL of the Lipase Assay Buffer in quintuplicate. The reaction mix was added to every well except for the standard.

Using the SpectraMax M5, the plate was shaken and incubated for 3 minutes prior to reading the relative fluorescence units (RFU) at λₑₓ/λₑₘ=529/600 nm. Incubation was continued at 37° C. and the microtiter plate was read every 5 minutes for 1 hour with shaking prior to every reading.

Abd-Elhakeem et al. (2013) developed a simple, rapid and precise colorimetric for determination of lipase activity in microbial media. The method is based on using phenyl acetate as substrate for lipase and determination of liberated phenol by Folin Ciocalteu reagent.

Following two methods are used with live bacteria or bacterial extracts without the need for purified enzymes:

A plate assay can be used to detect bacterial lipase by measuring rhodamine B fluorescence as described by Kouker & Jaaeger 1987. Presence of lipase causes rhodamine B to emit orange fluorescence that can be detected with UV light, a larger area of fluorescence indicating a higher concentration of lipase present.

0.001% wt/vol of rhodamine B and a lipase substrate, such as trioleoylglycerol or lipids from olive oil that have been purified by passage through a column, are added to nutrient agar during plate preparation. 20 mL of nutrient agar are poured into plastic petri dishes. P. aeruginosa, or strains such as S. aureus and S. epidermidis that are clinically relevant to blepharitis, are spread plated on the nutrient agar containing rhodamine B. Plates are incubated for 24-48 hours at 37° C., until a bacterial lawn forms. 3-8 mm diameter punches are made in the nutrient agar in preparation for lipase treatment.

10 μL of lipase or cell culture supernatant are added to the punches in the nutrient agar plate. Samples are treated with or without 0.001%-0.04% HOCl pH 4 or NaOCl pH 7 and 10. Lipase activity is measured by irradiating plates with 350 nm UV light and observing the area of orange fluorescence emission. If lipase has been inactivated by HOCl or NaOCl treatment, no fluorescence is detected.

A TLC assay for testing lipase activity was described by Dougherty McCulley, 1986. Aerobic bacteria are grown on 5% sheep blood agar plates for 24 hours. Anaerobic bacteria are grown for 72 hours on Brucella agar in an anaerobic gas-pak system. Bacterial strains are adjusted in saline to McFarland 1.0 (3.0×10⁸ CFU/mL). This McFarland adjusted suspension is used to inoculate test tubes containing various substrates in media. Substrates can include triglycerides, cholesterol ester, fatty wax, oleic acid, free cholesterol, cetyl alcohol, monoolein and diolean. Media containing no substrate are used as a control. Cultures are maintained for 7 days at 35° C. with daily vortexing. After incubation, each test tube is treated with 3×2 mL chloroform:methanol (3:1), dried with N₂, and dissolved in 50 μL chloroform.

Samples are run on a TLC plate consisting of hexane:diethyl ether:acetic acid (75:25:1). TLC plates are sprayed with a 1:1 solution of acetic acid and ethanol and heated to 100-150° C. for 10-30 minutes. TLC plates are observed for lipase activity against each substrate. Modifications can be made to the methods described in Dougherty McCulley, (1986) to detect lipase inactivation by HOCl and NaOCl.

Organisms can be grown in growth media without lipase substrates. After the incubation period, planktonic bacteria can be centrifuged out, leaving the bacterial lipase in the supernatant. The supernatant may be treated with or without 0.001%-0.04% HOCl pH 4 or NaOCl pH 7 and pH 10 followed by addition of lipase substrates and additional incubation at 35° C. The supernatant may be prepared as described above and run on TLC to determine if the lipase was inactivated.

Expression and purification of lipases are conducted as described in Simons et al., 1996. Recombinant S. aureus lipase may be isolated from E. coli by growing the organism in a fermenter at 37° C. with continuous stiffing, until optical density (OD) at 660 nm is 1.0-2.0. 0.4 mM isopropyl β-D-1-thiogalactopyranoside (IPTG) are added to the fermenter and incubated for an additional 240 hours. E. coli cells are collected by centrifuging at 5000×g for 20 minutes, at 4° C. Lipase are extracted at 4° C. from 100 g pelleted E. coli cells by re-suspending the pellet in 500 mL lysis buffer and homogenizing by sonication followed by centrifugation at 10,000×g for 30 minutes. 100 mM NaCl and 30 mL DEAE cellulose are added to the supernatant and stirred for 30 minutes to 2 hours. The suspension is passed through a filter and the filtrate is freeze dried.

Crude lipase samples are purified by column purification. Crude lipase are dissolved in 100 mL 6 M guanidine/HCl. Dissolved sample are dialysed with 10 mM Tris/HCl and 1 mM EDTA, pH 8.3, followed by centrifugation at 10,000×g for 10-30 minutes. A 175 mL DEAE cellulose column is washed with 10 mM Tris/HCl, pH 4 after loading the samples. Flow through are combined, adjusted to pH 6.5 with 1 M succinic acid, and loaded onto a 40 mL CM-cellulose column. A linear gradient of 0-1 M NaCl is used to elute recombinant S. aureus lipase from E. coli. These methods of isolation and purification can be modified for different lipase isozymes or lipases from different organisms such as S. aureus or S. epidermidis.

Test concentrations of HOCl and NaOCl range from 0.002% to 0.4%.

The test pH ranges from 4 to 10.

While the foregoing description describes specific embodiments, those with ordinary skill in the art will appreciate that various modifications and alternatives can be developed. Accordingly, the particular embodiments described above are meant to be illustrative only, and not to limit the scope of the invention, which is to be given the full breadth of the appended claims, and any and all equivalents thereof.

What is claimed is:

1. A method for the treatment or the prevention of blepharitis, meibomian gland dysfunction, or dry eye associated with lipases in a patient in need thereof, comprising an administration of a therapeutically effective amount of a pharmaceutical composition comprising hypochlorous acid, a hypochlorite salt or a mixtures thereof, to inactivate lipases.

2. The method of claim 1, wherein, wherein the hypochlorous acid, hypochlorite salt or a mixtures thereof is at a concentration of 0.005% to 0.05% in an aqueous saline solution.

3. The method of claim 2, wherein the concentration of the hypochlorous acid, hypochlorite salt or a mixtures thereof is 0.005% to 0.05% in a saline solution at a pH range of 3 to 9.

4. The method of claim 3, where the pH range is 3.5 to 4.5.

5. The method of claim 3, wherein the concentration of the hypochlorous acid, hypochlorite salt or a mixtures thereof is 0.01% in a saline solution at a pH of 4.

6. The method of claim 5, wherein the hypochlorite salt is NaOCl.

7. The method of claim 5, wherein the composition comprises hypochlorous acid at a concentration of 0.01% in a saline solution at pH 4.

8. The method of claim 7, wherein the pharmaceutical composition further comprises a disinfectant selected from the group consisting of chloramines, dichloroisocyanurate, trichloroisocyanurate, wet chlorine, chlorine dioxide and mixtures thereof.

9. The method of claim 8 wherein the lipases are from Burkholderia cepacia, Pseudomonas fluorescens, Thermus thermophilus, Talaromyces flavus and Burkholderia species.

10. The method of claim 9, wherein the lipases are inactivated in less than 10 minutes, less than 5 minutes, less than 2 minutes or less than 1 minute.

11. The method of claim 10, wherein the method reduces the activity of the lipases in the patient.

12. The method of claim 10, wherein the method reduces the activity of the lipases on human cells or tissues.

13. A method for reducing or eliminating the activity of lipases associated with blepharitis, meibomian gland dysfunction, or dry eye in a patient, the method comprising the administration of a therapeutically effective amount of a pharmaceutical composition comprising hypochlorous acid, a hypochlorite salt or mixtures thereof, to inactivate lipases.

14. The method of claim 13, wherein the inactivation of lipases is from bacterial species that are commensal (normal) skin flora or bacteria recovered from blepharitis, meibomian gland dysfunction or dry eye, or a combination thereof.

15. The method of claim 14, wherein the hypochlorous acid, hypochlorite salt or a mixtures thereof is at a concentration of 0.005% to 0.05% in an aqueous saline solution.

16. The method of claim 14, wherein the concentration of the hypochlorous acid, hypochlorite salt or a mixtures thereof is 0.005% to 0.05% in a saline solution at a pH range of 3 to 9.

17. The method of claim 16, wherein the concentration of the hypochlorous acid, hypochlorite salt or a mixtures thereof is 0.01% in a saline solution at a pH of 4.

18. The method of claim 17, wherein the hypochlorite salt is NaOCl.

19. The method of claim 18 wherein the lipases are from Burkholderiacepacia, Pseudomonas fluorescens, Thermus thermophilus, Talaromyces flavus and Burkholderias species.

20. The method of claim 19, wherein the method reduces the activity of the lipases on human cells or tissues.
